(19) United States
(12) Patent Application Publication
(10) Pub. No.: US 2021/0282817 A1
(43) Pub. Date: Sep. 16, 2021

(54) SPINAL FUSION SYSTEM AND SPINAL FUSION INSTRUMENT ADAPTER USED THEREIN

(71) Applicant: TEIJIN NAKASHIMA MEDICAL CO., ltd, Okayama (JP)

(72) Inventors: Yuichi Sato, Okayama (JP); Masato Otsubo Kanagawa, Kanagawa (JP); Tobimaru Takata, Tokyo (JP)

(21) Appl. No.: 16/481,063

(22) PCT Filed: Jan. 25, 2017
(86) PCT No.: PCT/JP2017/002518

§ 371 Date: Jul. 25, 2019

(51) Int. Cl. A61B 17/70

(52) U.S. Cl. CPC A61B 17/7032; A61B 17/7037

(57) ABSTRACT

A spinal fusion instrument adapter includes: an engaging portion protruding downward to engage with an engaged portion of a bone engagement member; and a restricting portion extending upward from the engaging portion and configured to, in a state where the engaging portion engages with the engaged portion of the bone engagement member in an accommodating portion of a head portion, directly or indirectly contacts an inner wall of the head portion to restrict three dimensional swinging motion of the bone engagement member, which is integrated with the spinal fusion instrument adapter.

### TECHNICAL FIELD

The present invention relates to a spinal fusion system and a spinal fusion instrument adapter used therein.

### BACKGROUND ART

There is a known spinal fusion technique in which, in order to keep the space between a plurality of vertebrae to reduce pressure on spinal nerves, a plurality of spinal fusion instruments, such as spinal screws, are engaged with the respective vertebrae, and a rod member is used to connect between the plurality of spinal fusion instruments. Each of the spinal fusion instruments used in the spinal fusion technique includes a bone engagement member and a head portion. The bone engagement member includes: a distal end portion, such as a screw portion, to be engaged with a bone; and a proximal end portion that includes, as at least a part thereof, a spherical surface. The head portion holds the proximal end portion of the bone engagement member.

Such spinal fusion instruments are categorized into a plurality of types, including a polyaxial type and a monoaxial type. The polyaxial-type spinal fusion instrument is configured such that, in a state where the rod member is not connected thereto, the bone engagement member is swingable three dimensionally relative to the head portion. The monoaxial-type spinal fusion instrument is configured such that, in a state where the rod member is not connected thereto, the bone engagement member is non-swingable relative to the head portion (see Patent Literature 1, for example).

### CITATION LIST

### Patent Literature

PTL 1: Japanese Patent No. 5883927

### SUMMARY OF INVENTION

### Technical Problem

Since the bone engagement member of the polyaxial-type spinal fusion instrument is freely movable relative to the head portion, the degree of freedom is high when performing the work of connecting the rod member to the head portion in a state where the bone engagement member is engaged with a vertebra. Thus, there is an advantage that the connecting work can be readily performed. However, there may arise a situation where even if the rod member is pre-deformed so that the arrangement of the vertebrae will be in an ideal shape, the spinal correction force applied by the rod member is reduced due to the degree of freedom of the head portion, resulting in that the spine cannot be corrected sufficiently. Meanwhile, since the monoaxial-type spinal fusion instrument does not have the aforementioned degree of freedom, there is a case where the work of connecting the rod member to the head portion of the monoaxial-type spinal fusion instrument is difficult. However, there is an advantage that the aforementioned problem in which the spinal correction force applied by the rod member is reduced due to the degree of freedom of the head portion does not occur, and the spine can be corrected sufficiently. Thus, each type of spinal fusion instrument has its own advantage and disadvantage. For this reason, in a spinal fusion surgery, the spinal fusion instrument to use is suitably selected from among these plurality of types of spinal fusion instruments.

Accordingly, when performing a spinal fusion surgery, it is necessary to prepare each of these plurality of types of bone engagement members in advance of the surgery. This means that a greater number of bone engagement members than what is actually necessary for performing the surgery are prepared in advance of the surgery. Therefore, there are problems, for example, the preparation takes time, and the inventory management becomes complex.

The present invention has been made to solve the above-described problems. An object of the present invention is to provide a spinal fusion system and a spinal fusion instrument adapter used therein, the spinal fusion system being usable as a plurality of types of spinal fusion instruments, thereby eliminating the need to prepare each of the plurality of types of spinal fusion instruments in advance.

### Solution to Problem

A spinal fusion instrument adapter according to one aspect of the present invention is a spinal fusion instrument adapter to be joined to a spinal fusion instrument. The spinal fusion instrument includes: a bone engagement member including a distal end portion to be engaged with a bone and a proximal end portion that includes, as at least a part thereof, a spherical surface; and a head portion configured to hold the proximal end portion of the bone engagement member, such that the bone engagement member is swingable three dimensionally with respect to a position of the proximal end portion. The bone engagement member includes an engaged portion that is open at a proximal end side of the proximal end portion. The head portion includes an inner wall demarcating an accommodating portion in which the proximal end portion of the bone engagement member is accommodatable between a lower end portion and an upper end portion of the head portion. The head portion is configured to accommodate the bone engagement member in the accommodating portion, such that the distal end portion of the bone engagement member is exposed from the lower end portion of the head portion. The spinal fusion instrument adapter includes: an engaging portion protruding downward to engage with the engaged portion; and a restricting portion extending upward from the engaging portion and configured to, in a state where the engaging portion engages with the engaged portion of the bone engagement member in the accommodating portion of the head portion, directly or indirectly contacts the inner wall of the head portion to restrict the three dimensional swinging motion of the bone engagement member, which is integrated with the spinal fusion instrument adapter.

According to the above configuration, in a state where the spinal fusion instrument adapter is not attached to the spinal fusion instrument, the bone engagement member is swingable three dimensionally relative to the head portion. On the other hand, in a state where the engaging portion of the spinal fusion instrument adapter engages with the engaged portion of the bone engagement member, and thereby the spinal fusion instrument adapter is fixed to the spinal fusion instrument, if the bone engagement member is about to swing, the swinging motion of the bone engagement member relative to the head portion is restricted since the restricting portion of the spinal fusion instrument adapter contacts the inner wall of the head portion. Thus, the mode of swinging of the bone engagement member relative to the head portion can be changed depending on whether or not to attach the spinal fusion instrument adapter to the spinal fusion instrument. Therefore, the spinal fusion instrument is usable as a plurality of types of spinal fusion instruments, thereby eliminating the need to prepare each of the plurality of types of spinal fusion instruments in advance.

The head portion may include: a head portion body including the inner wall; and a pressing member inserted in the accommodating portion and configured such that a lower end portion of the pressing member has a shape corresponding to the spherical surface of the proximal end portion of the bone engagement member. The pressing member may include an insertion hole in which the spinal fusion instrument adapter is insertable, the pressing member being configured to be in contact with the inner wall in the accommodating portion. The restricting portion may restrict the three dimensional swinging motion of the bone engagement member by contacting an inner wall of the insertion hole. In a state where the pressing member is in contact with the inner wall in the accommodating portion, as a result of the restricting portion of the spinal fusion instrument adapter coming into contact with the inner wall of the insertion hole of the pressing member, the restricting portion of the spinal fusion instrument adapter indirectly contacts the inner wall formed in the head portion body. Therefore, by bringing the engaging portion into engagement with the bone engagement member by inserting the spinal fusion instrument adapter into the insertion hole of the pressing member, the three dimensional swinging motion of the bone engagement member can be restricted assuredly.

The inner wall of the head portion may be formed cylindrically. An outer surface of the pressing member may be formed as a columnar surface that has an external diameter corresponding to the inner wall of the head portion. An outer surface of the restricting portion may be formed as a columnar surface that has an external diameter corresponding to an internal diameter of the insertion hole. Accordingly, the pressing member closely contacts the head portion, and also, the pressing member closely contacts the spinal fusion instrument adapter. Therefore, by attaching the spinal fusion instrument adapter to the spinal fusion instrument, the bone engagement member can be fixed substantially on a single axis.

A spinal fusion system according to another aspect of the present invention is a spinal fusion system including a spinal fusion instrument and a spinal fusion instrument adapter. The spinal fusion instrument includes: a bone engagement member including a distal end portion to be engaged with a bone and a proximal end portion that includes, as at least a part thereof, a spherical surface; and a head portion configured to hold the proximal end portion of the bone engagement member, such that the bone engagement member is swingable three dimensionally with respect to a position of the proximal end portion. The bone engagement member includes an engaged portion that is open at a proximal end side of the proximal end portion. The head portion includes an inner wall demarcating an accommodating portion in which the proximal end portion of the bone engagement member is accommodatable between a lower end portion and an upper end portion of the head portion, and the head portion is configured to accommodate the bone engagement member in the accommodating portion, such that the distal end portion of the bone engagement member is exposed from the lower end portion of the head portion. The spinal fusion instrument adapter includes: an engaging portion protruding downward to engage with the engaged portion; and a restricting portion extending upward from the engaging portion and configured to, in a state where the engaging portion engages with the engaged portion of the bone engagement member in the accommodating portion of the head portion, directly or indirectly contacts the inner wall of the head portion to restrict the three dimensional swinging motion of the bone engagement member, which is integrated with the spinal fusion instrument adapter.

The above object, other objects, features, and advantages of the present invention will be made clear by the following detailed description of a preferred embodiment with reference to the accompanying drawings.

### Advantageous Effects of Invention

The present invention is configured as described above, and provides an advantage of being usable as a plurality of types of spinal fusion instruments, thereby eliminating the need to prepare each of the plurality of types of spinal fusion instruments in advance.

### BRIEF DESCRIPTION OF DRAWINGS

FIG. 1 is a perspective view showing one example of a spinal fusion instrument used in a spinal fusion system of one embodiment of the present invention.

FIG. 2 is a perspective view showing a proximal end portion of a bone engagement member of the spinal fusion instrument of FIG. 1 and a spinal fusion instrument adapter to be engaged with the proximal end portion.

FIG. 3 shows a sectional view of the spinal fusion instrument of FIG. 1 and the spinal fusion instrument adapter, taken along the direction of an axis (longitudinal axis).

FIG. 4 shows a state where the spinal fusion instrument adapter of FIG. 3 engages with an engaged portion of the bone engagement member.

FIG. 5 shows a rod member being fixed in a state where the spinal fusion instrument adapter is attached to a head portion as shown in FIG. 4.

FIG. 6 is a perspective view showing another example of the spinal fusion instrument used in the spinal fusion system of the embodiment of the present invention.

FIG. 7A shows sectional views illustrating another example of the spinal fusion instrument and the spinal fusion instrument adapter used in the spinal fusion system of the embodiment of the present invention.

FIG. 7B shows sectional views illustrating yet another example of the spinal fusion instrument and the spinal fusion instrument adapter used in the spinal fusion system of the embodiment of the present invention.

### DESCRIPTION OF EMBODIMENTS

Hereinafter, an embodiment of the present invention is described with reference to the drawings. In the drawings, the same or corresponding elements are denoted by the same reference signs, and repeating the same descriptions is avoided below. In the description and the claims herein, the “upward” direction and the “downward” direction are determined based on the orientation of a spinal fusion instrument when a surgery is performed on a human body. That is, the lower side is defined as the closer side to the spine, and the upper side is defined as the farther side from the spine.

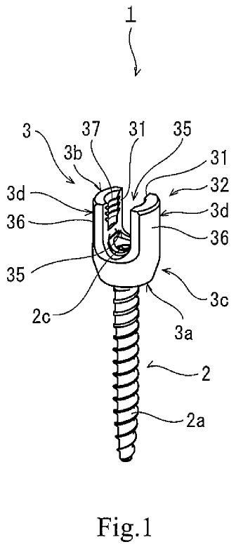

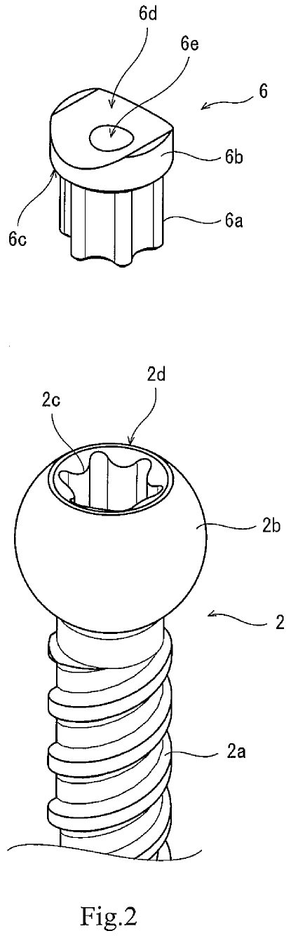

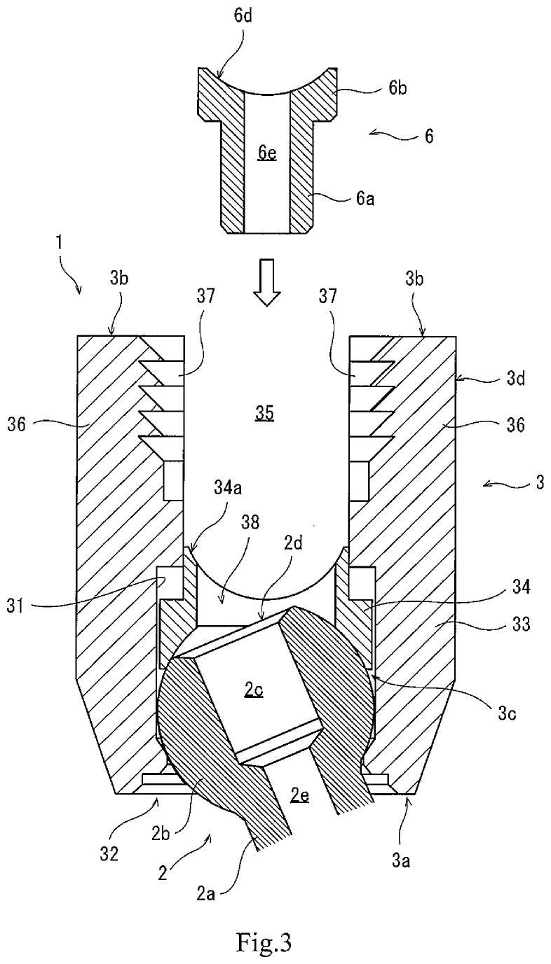

First, a spinal fusion instrument of one embodiment of the present invention is described. FIG. 1 is a perspective view showing one example of the spinal fusion instrument, which is used in a spinal fusion system of the embodiment of the present invention. FIG. 2 is a perspective view showing a proximal end portion of a bone engagement member of the spinal fusion instrument of FIG. 1 and a spinal fusion instrument adapter to be engaged with the proximal end portion. FIG. 3 shows a sectional view of the spinal fusion instrument of FIG. 1 and the spinal fusion instrument adapter, taken along the direction of an axis (longitudinal axis). As shown in FIG. 1, a spinal fusion instrument 1 includes: a bone engagement member 2 to be engaged with a bone, such as a vertebra; and a head portion 3 configured to hold a proximal end portion of the bone engagement member 2.

The bone engagement member 2 includes: a distal end portion 2a to be engaged with the bone; and a proximal end portion 2b including, as at least a part thereof, a spherical surface (see FIG. 2 and FIG. 3). In the example of FIG. 1, the distal end portion 2a of the bone engagement member 2 is configured as a screw shaft including a screw portion to be screwed into the bone, such as a vertebra. The bone engagement member 2 further includes an engaged portion 2c (see FIG. 2 and FIG. 3), which is open at the proximal end (upper end) side of the proximal end portion 2b. The engaged portion 2c includes a groove or a hole that has, for example, any of the following various shapes: a plus shape; a minus shape; a polygonal shape such as a hexagonal shape; and a hexalobular shape.

The bone engagement member 2 is held by the head portion 3, such that the bone engagement member 2 is rotatable about the axial direction relative to the head portion 3. The head portion 3 holds the proximal end portion 2b in such a manner that the bone engagement member 2 is swingable three dimensionally with respect to the position of the proximal end portion 2b of the bone engagement member 2. More specifically, the head portion 3 includes an inner wall 31 demarcating an accommodating portion 3c, in which the proximal end portion 2b of the bone engagement member 2 is accommodatable between a lower end portion 3a and upper end portions 3b of the head portion 3. The bone engagement member 2 is accommodated in the accommodating portion 3c, such that the distal end portion 2a of the bone engagement member 2 is exposed from the lower end portion 3a of the head portion 3.

The head portion 3 is formed substantially in the shape of a cylinder, and the inner wall 31 is formed such that a cross section thereof orthogonal to the axial direction of the cylinder has a circular shape. A protrusion hole 32, through which the distal end portion 2a of the bone engagement member 2 protrudes, is formed in a lower end portion of the accommodating portion 3c (i.e., the lower end portion 3a of the head portion 3). The lower end portion 3a of the head portion 3 has such a shape that the proximal end portion 2b of the bone engagement member 2 will not be pulled out of the protrusion hole 32. FIG. 3 shows a configuration example in which such a retaining shape is formed integrally with the body of the head portion 3. However, as an alternative, a retaining member may be fixed to a body 33 (described below) of the head portion 3. The protrusion hole 32 has a diameter (minimum value) that is less than the diameter of the spherical surface of the proximal end portion 2b of the bone engagement member 2.

Owing to the above-described configuration, in a state where the head portion 3 is held, by bringing a driver (not shown) having a projecting shape corresponding to the shape of the engaged portion 2c into engagement with the engaged portion 2c and rotating the driver about the axis, the bone engagement member 2 can be rotated relative to the head portion 3. In this manner, the bone engagement member 2 can be engaged with a predetermined vertebra, or the engagement of the bone engagement member 2 with the vertebra can be released.

The head portion 3 includes: the aforementioned head portion body 33 including the inner wall 31; and a pressing member 34 inserted in the accommodating portion 3c and configured such that a lower end portion of the pressing member 34 has a shape corresponding to the spherical surface of the proximal end portion 2b of the bone engagement member 2. In the present embodiment, the accommodating portion 3c is demarcated by the lower end portion of the pressing member 34 and the inner wall 31 of the head portion body 33, and the spherical surface of the proximal end portion 2b of the bone engagement member 2 slides on the lower end portion of the pressing member 34 and the inner wall 31 of the head portion body 33. As a result, the bone engagement member 2 swings three dimensionally. The three dimensional swinging means swinging of the bone engagement member 2 about two axes (X- and Y-axes) orthogonal to the axial direction (upward-downward direction, i.e., Z-axis) of the head portion 3. Thus, the spinal fusion instrument 1 is a polyaxial-type spinal fusion instrument whose bone engagement member 2 is swingable relative to the head portion 3 in any direction over 360° about the axis of the head portion 3 to such a position that the angle between the axis of the bone engagement member 2 and the axis of the head portion 3 becomes a predetermined angle (e.g.,) 30°.

The head portion 3 (specifically the head portion body 33) includes an extension portion 3d extending upward from the accommodating portion 3c. A pair of slots 35 is formed in the extension portion 3d, such that each slot 35 is radially open at a part of the circumference of the extension portion 3d. Each of the pair of slots 35 is formed such that the upper end of the extension portion 3d is open and the lower end of the extension portion 3d is arc-shaped. As a result of the pair of slots 35 being formed, the extension portion 3d is configured to include a pair of arc-shaped portions 36 facing each other and extending upward from the accommodating portion 3c.

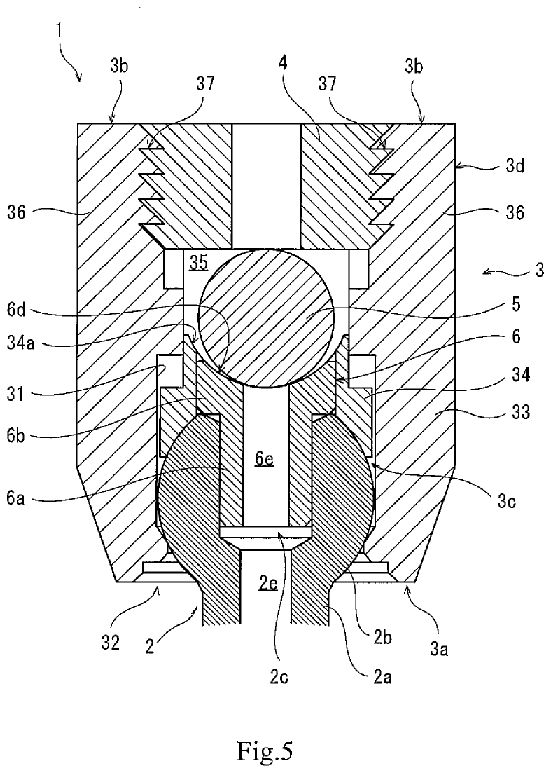

The inner wall of each of the pair of arc-shaped portions 36 is provided with a thread groove 37, into which a set screw 4 (see FIG. 5 referred to below) can be screwed. An upper end portion 34a of the pressing member 34 includes a curved surface such that, when seen in a predetermined cross section (e.g., the cross section of FIG. 3) including the axial direction of the head portion 3, the curved surface forms a downward-projecting arc shape and has a diameter corresponding to the external diameter of a rod member 5 (see FIG. 5 referred to below). The pressing member 34 is configured to be slidable in the upward-downward direction (axial direction) in the accommodating portion 3c. To be more specific, the pressing member 34 is formed to have a cylindrical shape, and the external diameter of the pressing member 34 corresponds to the internal diameter of the inner wall 31 of the head portion body 33.

In the above-described configuration, the rod member 5 is placed on the pressing member 34 in the accommodating portion 3c through the pair of slots 35, and then the set screw 4 is screwed into the thread grooves 37. As a result, the set screw 4 presses the pressing member 34 and the proximal end portion 2b of the bone engagement member 2 via the rod member 5, and thereby the rod member 5 and the bone engagement member 2 are fixed.

Thus, even in the case of the polyaxial-type spinal fusion instrument 1 whose bone engagement member 2 is swingable three dimensionally relative to the head portion 3, after the rod member 5 is fixed by using the set screw 4, the bone engagement member 2 becomes unable to swing relative to the head portion 3. However, at the time of fixing the rod member 5, the head portion 3 may swing relative to the bone engagement member 2, making it difficult to keep the head portion 3 in a suitable orientation. If such relative swinging motion of the head portion 3 has a significantly negative impact, it is necessary to restrict the swinging motion of the bone engagement member 2 relative to the head portion 3 in advance.

In this respect, the spinal fusion system of the present embodiment includes a spinal fusion instrument adapter 6 for restricting the three dimensional swinging motion. The spinal fusion instrument adapter 6 includes an engaging portion 6a and a restricting portion 6b. The engaging portion 6a protrudes downward to engage with the engaged portion 2c of the bone engagement member 2. The restricting portion 6b extends upward from the engaging portion 6a. In a state where the engaging portion 6a engages with the engaged portion 2c of the bone engagement member 2 in the accommodating portion 3c of the head portion 3, the restricting portion 6b directly or indirectly contacts the inner wall 31 of the head portion 3 to restrict three dimensional swinging motion of the bone engagement member 2, which is integrated with the spinal fusion instrument adapter 6. In the example shown in FIG. 2, the engaged portion 2c formed in the proximal end portion 2b of the bone engagement member 2 includes a hole in a hexalobular shape. In accordance therewith, the engaging portion 6a of the spinal fusion instrument adapter 6 is formed as a projecting portion having a hexalobular shape. The restricting portion 6b is formed to have a substantially columnar shape, and extends from the upper end of the engaging portion 6a in a direction (horizontal direction) orthogonal to the axial direction of the engaging portion 6a.

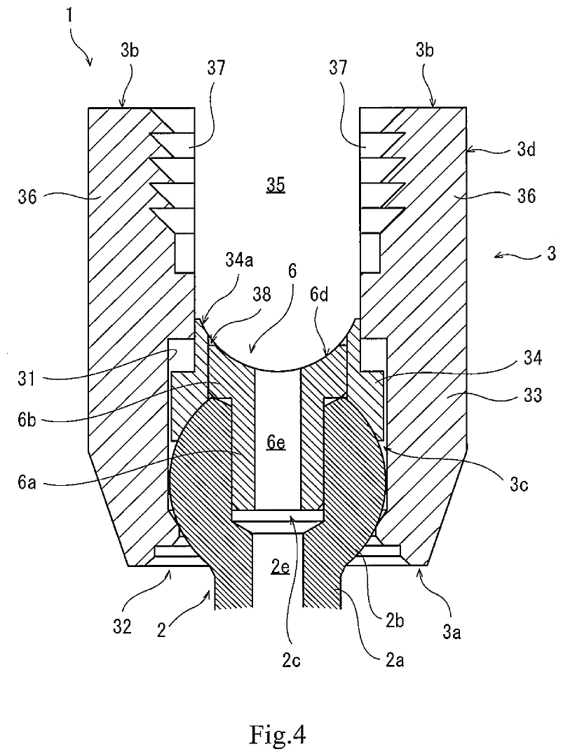

As shown in FIG. 3, when bringing the spinal fusion instrument adapter 6 into engagement with the engaged portion 2c of the bone engagement member 2, the spinal fusion instrument adapter 6 is inserted into the accommodating portion 3c through the upper end portions 3b of the head portion 3. FIG. 4 shows a state where the spinal fusion instrument adapter of FIG. 3 engages with the engaged portion of the bone engagement member. In the present embodiment, the pressing member 34 includes an insertion hole 38, in which the spinal fusion instrument adapter 6 is insertable. The insertion hole 38 has a circular cross section, and the diameter of the insertion hole 38 corresponds to the external diameter of the restricting portion 6b of the spinal fusion instrument adapter 6. Accordingly, the restricting portion 6b is slidable in the insertion hole 38 of the pressing member 34 in the upward-downward direction.

By bringing the engaging portion 6a into engagement with the engaged portion 2c of the proximal end portion 2b of the bone engagement member 2 in a manner to insert the spinal fusion instrument adapter 6 into the insertion hole 38 of the pressing member 34, the outer surface of the restricting portion 6b comes into contact with the inner wall of the pressing member 34, the inner wall demarcating the insertion hole 38. As mentioned above, the external diameter of the pressing member 34 corresponds to the internal diameter of the inner wall 31 of the head portion body 33. Accordingly, when the spinal fusion instrument adapter 6 is integrated with the bone engagement member 2, the restricting portion 6b is in such a state that the entire circumference of the restricting portion 6b is in contract with the inner wall 31 of the head portion body 33 via the pressing member 34. Thus, as a result of attaching the spinal fusion instrument adapter 6 to the spinal fusion instrument 1, the spinal fusion instrument 1 can function as a monoaxial-type spinal fusion instrument whose bone engagement member 2 is substantially fixed on a single axis. In the present embodiment, when the spinal fusion instrument adapter 6 is attached to the spinal fusion instrument 1, the axis of the bone engagement member 2 is not substantially shifted from the axis of the head portion 3.

It should be noted that the axis of the bone engagement member 2 being not substantially shifted from the axis of the head portion 3 means that the axis of the bone engagement member 2 is not at a position at which an angle formed by the axis of the bone engagement member 2 and the axis of the head portion 3 exceeds a tolerable angular error range (e.g., less than 5°). In other words, the head portion 3 and the bone engagement member 2 need not be fully integrated together by the spinal fusion instrument adapter 6.

As thus described, according to the above configuration, in a state where the spinal fusion instrument adapter 6 is not attached to the spinal fusion instrument 1, the bone engagement member 2 is swingable three dimensionally relative to the head portion 3. On the other hand, in a state where the engaging portion 6a of the spinal fusion instrument adapter 6 engages with the engaged portion 2c of the bone engagement member 2, and thereby the spinal fusion instrument adapter 6 is fixed to the spinal fusion instrument 1, if the bone engagement member 2 is about to swing, the swinging motion of the bone engagement member 2 relative to the head portion 3 is restricted since the restricting portion 6b of the spinal fusion instrument adapter 6 contacts the inner wall 31 of the head portion 3. Thus, the mode of swinging of the bone engagement member 2 relative to the head portion 3 can be changed depending on whether or not to attach the spinal fusion instrument adapter 6 to the spinal fusion instrument 1. Therefore, the spinal fusion instrument 1 is usable as a plurality of types of spinal fusion instruments, thereby eliminating the need to prepare each of the plurality of types of spinal fusion instruments in advance.

Further, in the present embodiment, it is not necessary to machine the existing polyaxial-type spinal fusion instrument 1 in order to attach the spinal fusion instrument adapter 6 to the spinal fusion instrument 1. Therefore, a plurality of modes of swinging can be readily realized by applying the spinal fusion instrument adapter 6 to the existing spinal fusion instrument 1.

In the present embodiment, the outer surface of the pressing member 34 is formed as a columnar surface that has an external diameter corresponding to the inner wall 31, which is formed cylindrically in the head portion body 33, and the outer surface of the restricting portion 6b of the spinal fusion instrument adapter 6 is formed as a columnar surface that has an external diameter corresponding to the internal diameter of the insertion hole 38 of the pressing member 34. Accordingly, the pressing member 34 closely contacts the head portion body 33, and also, the pressing member 34 closely contacts the spinal fusion instrument adapter 6. Therefore, by attaching the spinal fusion instrument adapter 6 to the spinal fusion instrument 1, three dimensional swinging motion of the bone engagement member 2 can be restricted stably.

Further, in the present embodiment, the pressing member 34 is brought into contact with the inner wall 31 in the accommodating portion 3c, and in this state, the spinal fusion instrument adapter 6 is configured to be slidable on the pressing member 34 in the upward-downward direction in the insertion hole 38 of the pressing member 34. Accordingly, the outer surface of the restricting portion 6b of the spinal fusion instrument adapter 6 indirectly contacts the inner wall 31 formed in the head portion body 33. Therefore, by bringing the engaging portion 6a into engagement with the bone engagement member 2 by inserting the spinal fusion instrument adapter 6 into the insertion hole 38 of the pressing member 34, three dimensional swinging motion of the bone engagement member 2 can be restricted assuredly.

In the present embodiment, the engaged portion 2c of the proximal end portion 2b of the bone engagement member 2 includes an end surface 2d, which is parallel to a plane orthogonal to the axial direction. The restricting portion 6b of the spinal fusion instrument adapter 6 includes a lower surface 6c (connected to the engaging portion 6a), and the lower surface 6c is a flat surface facing the end surface 2d of the bone engagement member 2. Accordingly, the spinal fusion instrument adapter 6 can be stably engaged with the bone engagement member 2.

FIG. 5 shows the rod member being fixed in a state where the spinal fusion instrument adapter is attached to the head portion as shown in FIG. 4. As shown in FIG. 5, also in a case where the spinal fusion instrument adapter 6 is attached to the spinal fusion instrument 1 (i.e., a case where the spinal fusion instrument 1 functions as a monoaxial-type spinal fusion instrument), similar to a case where the spinal fusion instrument adapter 6 is not attached to the spinal fusion instrument 1 (i.e., a case where the spinal fusion instrument 1 functions as a polyaxial-type spinal fusion instrument), the rod member 5 is placed on the pressing member 34 and/or the spinal fusion instrument adapter 6 through the pair of slots 35, and then the set screw 4 is screwed into the thread grooves 37. As a result, the set screw 4 presses the spinal fusion instrument adapter 6, the pressing member 34, and the proximal end portion 2b of the bone engagement member 2 via the rod member 5, and thereby the rod member 5 and the bone engagement member 2 are fixed.

In the present embodiment, an upper end portion 6d of the spinal fusion instrument adapter 6 (specifically, the upper end portion 6d of the restricting portion 6b) includes a curved surface such that, when seen in a predetermined cross section (e.g., the cross section of FIG. 3) including the axial direction of the head portion 3, the curved surface forms a downward-projecting arc shape and has a diameter corresponding to the external diameter of the rod member 5. The length of the spinal fusion instrument adapter 6 in the upward-downward direction is set such that, in a state where the engaging portion 6a engages with the engaged portion 2c of the bone engagement member 2, the upper surface of the spinal fusion instrument adapter 6 forms the same surface (curved surface) together with the upper surface of the pressing member 34. Accordingly, the rod member 5 can be received by both the upper end portion 34a of the pressing member 34 and the upper end portion 6d of the spinal fusion instrument adapter 6, and thereby the rod member 5 can be held stably.

The bone engagement member 2 includes a first guide hole 2e, which extends in the axial direction. The spinal fusion instrument adapter 6 includes a second guide hole 6e, which is formed at a position corresponding to the first guide hole 2e and extends in a direction in which the engaging portion 6a extends.

The bone engagement member 2 of the spinal fusion instrument 1 is fixed to a vertebra in the following steps. First, a skin incision is made above a predetermined part of the vertebra to which the spinal fusion instrument 1 is to be engaged. Desirably, the skin incision is made over a region that is substantially the same as a projection region when the spinal fusion instrument 1 is seen in a plan view (i.e., seen in the axial direction). That is, it is desirable that the skin incision region be substantially the same as (slightly greater than) the maximum diameter of the head portion 3 and/or an extender for holding the head portion 3. Thereafter, a thread groove corresponding to the screw diameter of the distal end portion 2a of the bone engagement member 2 is formed in the exposed vertebra by using a thread forming tool, such as one called a tap. Thereafter, through the skin incision region, the spinal fusion instrument 1 is inserted into the thread groove formed in the vertebra. At the time, through the skin incision region, a guide wire having an external diameter smaller than the internal diameter of the first guide hole 2e is inserted into the thread groove in the vertebra. The spinal fusion instrument 1 is guided by the inserted guide wire in such a manner that the guide wire is inserted through the first guide hole 2e of the bone engagement member 2, and thereby the spinal fusion instrument 1 is introduced into the body of the patient through the skin incision region. Thereafter, in a state where the spinal fusion instrument 1 is introduced in the patient's body, a driver is brought into engagement with the engaged portion 2c of the bone engagement member 2, and the bone engagement member 2 is rotated about the axis via the driver. In this manner, the bone engagement member 2 is engaged with the thread groove in the vertebra.

Further, in order to cause the spinal fusion instrument 1 introduced in the patient's body to function as a monoaxial-type spinal fusion instrument, a guide wire is again inserted into the first guide hole 2e. In this state, the spinal fusion instrument adapter 6 is introduced into the accommodating portion 3c of the head portion 3 in such a manner that the guide wire is inserted through the second guide hole 6e of the spinal fusion instrument adapter 6, and the engaging portion 6a is brought into engagement with the engaged portion 2c of the bone engagement member 2. Thus, since the second guide hole 6e, which allows the guide wire to be inserted therethrough, is formed in the spinal fusion instrument adapter 6, the mode of swinging can be changed without causing more damage than necessary to the patient.

Accordingly, the second guide hole 6e may have any shape, for example, a circular or polygonal shape, so long as the second guide hole 6e has such a size that the guide wire can be smoothly passed through the second guide hole 6e.

Alternatively, the second guide hole 6e may have such a shape that the second guide hole 6e is engaged with the driver non-rotatably about the axis. In this case, first, outside the patient's body, the engaging portion 6a of the spinal fusion instrument adapter 6 may be brought into engagement with the engaged portion 2c of the bone engagement member 2 non-rotatably about the axis, and then the spinal fusion instrument 1 may be inserted through the skin incision region into the thread groove formed in the vertebra. In a state where the spinal fusion instrument 1 and the spinal fusion instrument adapter 6 are introduced in the patient's body, the driver is brought into engagement with the second guide hole 6e of the spinal fusion instrument adapter 6, and the bone engagement member 2 is rotated about the axis via the driver and the spinal fusion instrument adapter 6. In this manner, the bone engagement member 2 can be engaged with the thread groove in the vertebra.

There may be a case where, first, outside the patient's body, the engaging portion 6a of the spinal fusion instrument adapter 6 is brought into engagement with the engaged portion 2c of the bone engagement member 2, and thereby the spinal fusion instrument adapter 6 and the head portion 3 are press-fitted together such that the spinal fusion instrument adapter 6 and the head portion 3 are non-rotatable about the axis relative to each other. In this case, the second guide hole 6e may be eliminated. In this case, a predetermined jig may be attached to the head portion 3 of the spinal fusion instrument 1 introduced in the patient's body, such that the jig and the head portion 3 are non-rotatable about the axis relative to each other. Then, the jig may be rotated about the axis to rotate the bone engagement member 2 about the axis via the head portion 3 and the spinal fusion instrument adapter 6, and thereby the bone engagement member 2 may be engaged with the thread groove in the vertebra. In a case where the spinal fusion instrument adapter 6 and the head portion 3 are not sufficiently press-fitted together, the predetermined jig may be configured to, when attached to the head portion 3, press the pressing member 34 to the proximal end portion 2b of the bone engagement member 2, and thereby the head portion 3 and the bone engagement member 2 may be rendered non-rotatable about the axis relative to each other.

Although the embodiment of the present invention has been described above, the present invention is not limited to the above embodiment, and various improvements, alterations, and modifications can be made to the above embodiment without departing from the spirit of the present invention.

For example, the above embodiment describes an example in which the spinal fusion instrument adapter 6 is applied to the spinal fusion instrument 1 whose head portion 3 is provided with no pair of tabs extending therefrom. However, the spinal fusion instrument adapter 6 may be applied also to the spinal fusion instrument whose head portion 3 is provided with a pair of tabs extending therefrom.

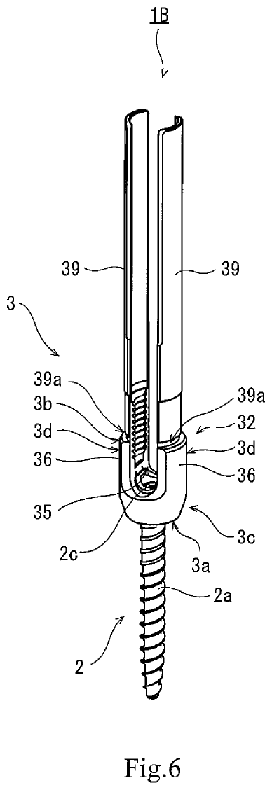

FIG. 6 is a perspective view showing another example of the spinal fusion instrument used in the spinal fusion system of the embodiment of the present invention. In the example of FIG. 6, the same components as those described in the example of FIG. 1 are denoted by the same reference signs as those used in the example of FIG. 1, and repeating the same descriptions is avoided below. FIG. 6 shows a spinal fusion instrument 1B, which is different from the spinal fusion instrument 1 of FIG. 1 in the following point: the spinal fusion instrument 1B includes a pair of tabs 39, each of which extends in the axial direction from the axial distal end of the extension portion 3d of the head portion 3. Each of the pair of tabs 39 extends upward from the upper end of a corresponding one of the pair of arc-shaped portions 36. The pair of tabs 39 has a predetermined internal diameter.

The pair of tabs 39 has such a length that, in a state where the bone engagement member 2 is engaged with a bone of a patient, the tabs 39 extend outward from the patient's skin (through the opening of a skin incision region). During a surgery, the pair of tabs 39 serves as a passage for the rod member 5, the set screw 4, or the like, which will be described below. The pair of tabs 39 is formed such that connecting portions 39a thereof, which connect to the pair of arc-shaped portions 36, are thinner than the other portions of the pair of tabs 39. By bending the pair of tabs 39 at the connecting portions 39a, the pair of tabs 39 can be cut away from the extension portion 3d. Alternatively, the pair of tabs 39 may be configured such that predetermined extension members are attachable to the outer surfaces of the tabs 39. The extension members may have such a length that, in a state where the extension members are attached to the pair of tabs 39 and the bone engagement member 2 is engaged with the patient's bone, the extension members extend outward from the patient's skin. During a surgery, the extension members may serve as a passage for the rod member 5, the set screw 4, or the like. In this case, the pair of tabs 39 may have a shorter length than in a case where the pair of tabs 39 alone forms the passage for the rod member 5, the set screw 4, or the like during the surgery.

In the above-described embodiment, the spinal fusion instrument adapter 6 is configured to come into indirect contact with the inner wall 31 of the head portion 3 (specifically, the inner wall 31 of the head portion body 33) via the pressing member 34. However, this example is non-limiting. Alternatively, the restricting portion 6b of the spinal fusion instrument adapter 6 may come into direct contact with the inner wall 31 of the head portion 3. For example, the spinal fusion instrument adapter 6 is applicable also to a configuration from which the pressing member 34 is eliminated.

The spinal fusion instrument is not limited to the spinal fusion instruments 1 and 1B of the above-described embodiment, but may be configured in various manners. For example, in the above-described embodiment, the set screw 4 is configured to be screwed into the thread grooves 37 formed in the inner wall 31 of the head portion 3. However, as an alternative, the set screw 4 may be fitted to the extension portion 3d of the head portion 3 via an intermediate member such as a locking cap. In this case, the inner wall 31 of the extension portion 3d of the head portion 3 is provided with an engagement portion (e.g., a recess or a projection) to be engaged with the intermediate member. The intermediate member has a cylindrical shape, and the outer side of the intermediate member has a shape to be engaged with the engagement portion of the inner wall 31. A thread groove is formed on the inner wall of the intermediate member so that the set screw 4 can be screwed into the thread groove.

The shape of the head portion 3 of the spinal fusion instrument is not limited to the cylindrical shape described in the above embodiment. For example, the head portion 3 may have a rectangular cylindrical shape with a polygonal cross section, or may have such a shape that the outer periphery of the cross section includes curved portions and straight portions (e.g., the shape of a square tube with round corners). Engaged portions, such as grooves, to be engaged with a holding member may be formed on the upper end portions 3b of the head portion 3.

In the above-described embodiment, the restricting portion 6b of the spinal fusion instrument adapter 6 contacts the inner wall 31 of the head portion 3 via the pressing member 34 over the entire circumference about the axis. However, as an alternative, over the circumference about the axis, there may be positions where the restricting portion 6b does not contact the inner wall 31 (i.e., there may be positions where gaps are present). That is, in a state where the engaging portion 6a of the spinal fusion instrument adapter 6 engages with the engaged portion 2c of the bone engagement member 2, if the bone engagement member 2 is about to swing at least in one predetermined direction, the restricting portion 6b is only required to directly or indirectly contact at least one part of the inner wall 31 of the head portion 3.

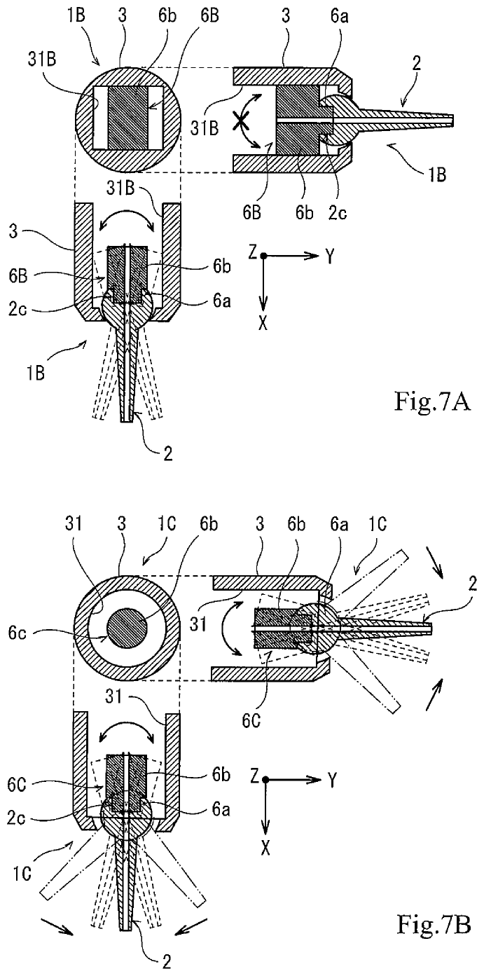

FIG. 7A and FIG. 7B are sectional views showing other examples of the spinal fusion instrument and the spinal fusion instrument adapter used in the spinal fusion system of the embodiment of the present invention. In each of FIG. 7A and FIG. 7B, the upper left drawing shows a Z-axial sectional view (a sectional view taken along an X-Y plane) orthogonal to the axis; the drawing on the right shows a sectional view taken along a Z-X plane; and the drawing on the bottom shows a sectional view taken along a Y-Z plane. FIG. 7A and FIG. 7B show examples in which the spinal fusion instrument adapter directly contacts the inner wall 31 of the head portion 3. However, alternatively, also in these examples, the spinal fusion instrument adapter may indirectly contact the inner wall 31 via the pressing member 34.

The spinal fusion instrument 1B in the example of FIG. 7A includes four flat surfaces, such that the cross section of an inner wall 31B of the head portion 3 orthogonal to the axis is a quadrangle. The restricting portion 6b of a spinal fusion instrument adapter 6B has such a shape that the restricting portion 6b is always in contact with, among the four flat surfaces of the inner wall 31, any two flat surfaces facing each other (in this example, the two flat surfaces facing each other in the X-axis direction). Accordingly, in a state where the engaging portion 6a of the spinal fusion instrument adapter 6B engages with the engaged portion 2c of the bone engagement member 2, the spinal fusion instrument 1B, which is originally swingable three dimensionally, is allowed to swing in the Y-Z plane, but swinging motion of the spinal fusion instrument 1B in the other directions is restricted. That is, the spinal fusion instrument 1B with the spinal fusion instrument adapter 6B attached thereto functions as a uniplanar-type spinal fusion instrument whose bone engagement member 2 swings in one predetermined plane.

A spinal fusion instrument 1C in the example of FIG. 7B is not different in structure from the spinal fusion instrument 1 of the above-described embodiment, and includes the cylindrical inner wall 31. The restricting portion 6b of a spinal fusion instrument adapter 6C is configured such that, in a state where the axis of the spinal fusion instrument adapter 6C coincides with the axis of the head portion 3, a predetermined gap is formed between the outer surface of the restricting portion 6b and the inner wall 31. In the example of FIG. 7B, the external diameter of the restricting portion 6b is smaller than the internal diameter of the inner wall 31 of the head portion 3. Accordingly, even in a state where the engaging portion 6a of the spinal fusion instrument adapter 6C engages with the engaged portion 2c of the bone engagement member 2, the spinal fusion instrument 1C is allowed to swing three dimensionally. However, the range of the swinging motion is restricted to a narrower range by the spinal fusion instrument adapter 6C. For example, assume that the swing angle is 0° when the axis of the bone engagement member 2 coincides with the axis of the head portion 3. In this case, when the spinal fusion instrument adapter 6C is not attached, the bone engagement member 2 can swing to an angular position of about 30°, whereas when the spinal fusion instrument adapter 6C is attached, the bone engagement member 2 can swing only to an angular position of about 10°. In this manner, the range of the swinging motion may be restricted to a narrower range by the spinal fusion instrument adapter 6C.

### INDUSTRIAL APPLICABILITY

The spinal fusion system and the spinal fusion instrument adapter used therein according to the present invention are useful since they are usable as a plurality of types of spinal fusion instruments, thereby eliminating the need to prepare each of the plurality of types of spinal fusion instruments in advance.

### REFERENCE SIGNS LIST

1, 1B spinal fusion instrument

2 bone engagement member

2a distal end portion

2b proximal end portion

2c engaged portion

3 head portion

3c accommodating portion

6 spinal fusion instrument adapter

6a engaging portion

6b restricting portion

31 inner wall

33 head portion body

34 pressing member

38 insertion hole

1. A spinal fusion instrument adapter to be joined to a spinal fusion instrument, the spinal fusion instrument including: a bone engagement member including a distal end portion to be engaged with a bone and a proximal end portion that includes, as at least a part thereof, a spherical surface; and a head portion configured to hold the proximal end portion of the bone engagement member, such that the bone engagement member is swingable three dimensionally with respect to a position of the proximal end portion,
the bone engagement member including an engaged portion that is open at a proximal end side of the proximal end portion,
the head portion including an inner wall demarcating an accommodating portion in which the proximal end portion of the bone engagement member is accommodatable between a lower end portion and an upper end portion of the head portion, the head portion being configured to accommodate the bone engagement member in the accommodating portion, such that the distal end portion of the bone engagement member is exposed from the lower end portion of the head portion,
the spinal fusion instrument adapter comprising:
an engaging portion protruding downward to engage with the engaged portion; and
a restricting portion extending upward from the engaging portion and configured to, in a state where the engaging portion engages with the engaged portion of the bone engagement member in the accommodating portion of the head portion, directly or indirectly contacts the inner wall of the head portion to restrict the three dimensional swinging motion of the bone engagement member, which is integrated with the spinal fusion instrument adapter.

2. The spinal fusion instrument adapter according to claim 1, wherein
the head portion includes:
a head portion body including the inner wall; and
a pressing member inserted in the accommodating portion and configured such that a lower end portion of the pressing member has a shape corresponding to the spherical surface of the proximal end portion of the bone engagement member,
the pressing member includes an insertion hole in which the spinal fusion instrument adapter is insertable, the pressing member being configured to be in contact with the inner wall in the accommodating portion, and
the restricting portion restricts the three dimensional swinging motion of the bone engagement member by contacting an inner wall of the insertion hole.

3. The spinal fusion instrument adapter according to claim 2, wherein
the inner wall of the head portion is formed cylindrically,
an outer surface of the pressing member is formed as a columnar surface that has an external diameter corresponding to the inner wall of the head portion, and
an outer surface of the restricting portion is formed as a columnar surface that has an external diameter corresponding to an internal diameter of the insertion hole.

4. A spinal fusion system comprising:
a spinal fusion instrument; and
a spinal fusion instrument adapter, wherein
the spinal fusion instrument includes:
a bone engagement member including a distal end portion to be engaged with a bone and a proximal end portion that includes, as at least a part thereof, a spherical surface; and
a head portion configured to hold the proximal end portion of the bone engagement member, such that the bone engagement member is swingable three dimensionally with respect to a position of the proximal end portion,
the bone engagement member includes an engaged portion that is open at a proximal end side of the proximal end portion,
the head portion includes an inner wall demarcating an accommodating portion in which the proximal end portion of the bone engagement member is accommodatable between a lower end portion and an upper end portion of the head portion, and the head portion is configured to accommodate the bone engagement member in the accommodating portion, such that the distal end portion of the bone engagement member is exposed from the lower end portion of the head portion, and
the spinal fusion instrument adapter includes:
an engaging portion protruding downward to engage with the engaged portion; and
a restricting portion extending upward from the engaging portion and configured to, in a state where the engaging portion engages with the engaged portion of the bone engagement member in the accommodating portion of the head portion, directly or indirectly contacts the inner wall of the head portion to restrict the three dimensional swinging motion of the bone engagement member, which is integrated with the spinal fusion instrument adapter.
